(19) United States
(12) Patent Application Publication
(10) Pub. No.: US 2025/0268683 A1
(43) Pub. Date: Aug. 28, 2025

(54) TRANSSCLERAL ILLUMINATOR

(71) Applicant: Alcon Inc., Fribourg (CH)

(72) Inventor: Paul R. Hallen, Colleyville, TX (US)

(21) Appl. No.: 19/023,702

(22) Filed: Jan. 16, 2025

Related U.S. Application Data
(60) Provisional application No. 63/556,499, filed on Feb. 22, 2024.

(51) Int. Cl. A61B 90/30; A61F 9/007

(52) U.S. Cl. CPC A61B 90/30; A61F 9/007; A61B 2090/306

(57) ABSTRACT

A transscleral illuminator includes a base and an optical fiber. The base has a geometry that is based on at least two cannulas inserted in a pars plana of a human eye. The base includes an inferior surface and a superior surface. The inferior surface is configured to interface with an external portion of the human eye. The optical fiber is disposed in the base and configured to transmit light through the base and the external portion of the human eye to illuminate an internal portion of the human eye.

### BACKGROUND

Anatomically, the human eye is divided into two distinct regions: the anterior segment and the posterior segment. The anterior segment includes the lens and extends from the outermost layer of the cornea to the posterior of the lens capsule. The posterior segment of the eye includes the anterior hyaloid membrane and all of the ocular structures behind it, such as the vitreous humor, retina, choroid, and the optic nerve.

Vitreoretinal surgery is performed within the posterior segment of the human eye to treat serious conditions including, but not limited to, age-related macular degeneration (AMD), diabetic vitreous hemorrhage, macular holes, retinal detachment, epiretinal membrane, diabetic retinopathy, and cytomegalovirus retinitis. Such procedures frequently require the severance and removal of portions of the vitreous humor from the posterior segment of the eye. The vitreous humor is a colorless, gel-like substance made of water, collagen, and hyaluronic acid.

Vitreoretinal surgery may require incisions and insertion of surgical instruments within an eye to repair the retina and/or perform portions of various surgical procedures. In order to visualize the retina and the surgical instruments within the eye, it is important that enough light is available to illuminate the surgical site. This light is typically provided via an endo-illuminator which is inserted through a first cannula and then manipulated by a surgeon with one hand. Simultaneously, a particular surgical instrument is inserted through a second cannula and then manipulated by the surgeon with the other hand.

There have been improvements in the art, such as improved manufacturing technology, that have significantly reduced the size (an outer diameter) of the surgical instruments utilized to perform vitreoretinal surgery; however, this reduction in size has also reduced the size (the outer diameter) of endo-illuminator optical fibers which limits the amount of light available to illuminate the surgical site. Therefore, improved illumination instruments and techniques for illumination during vitreoretinal surgery are desirable.

### SUMMARY

Aspects of the present disclosure relate to ophthalmic (eye) procedures, and more specifically, to illumination for visualization during ophthalmic procedures.

In certain embodiments, a device is provided. The device includes a base having an inferior surface and a superior surface and an optical fiber disposed in the base. The inferior surface is configured to interface with an external portion of a human eye. The base has a geometry that is based on at least two cannulas inserted in a pars plana of the human eye. The optical fiber is configured to transmit light through the base and the external portion of the human eye to illuminate an internal portion of the human eye.

In certain embodiments, a transscleral illuminator includes a base having a first surface and a second surface and at least one optical fiber partially disposed within the base. The first surface is configured to contact a sclera of a human eye. The base has an adjustable geometry. The at least one optical fiber is configured to transmit light from a light source through the first surface and the sclera to illuminate an inner portion of the human eye.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a representation of a human eye, according to embodiments described herein.

FIGS. 2A, 2B, 2C, 2D, and 2E illustrate top views of transscleral illuminator examples, according to embodiments described herein.

FIGS. 3A and 3B illustrate top views of optical fiber interface orientation examples, according to embodiments described herein.

FIGS. 4A and 4B illustrate cross-sectional views of transscleral illuminator examples, according to embodiments described herein.

FIGS. 5A and 5B illustrate cross-sectional views of transscleral illuminator examples relative to a representation of a human eye, according to embodiments described herein.

FIGS. 6A, 6B, and 6C illustrate a first example of an adjustable geometry of a transscleral illuminator, according to embodiments described herein.

FIGS. 7A, 7B, and 7C illustrate a second example of an adjustable geometry of a transscleral illuminator, according to embodiments described herein.

FIGS. 8A, 8B, and 8C illustrate a third example of an adjustable geometry of a transscleral illuminator, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to ophthalmic diagnostics and procedures, and more specifically, to transscleral illumination for visualization of an internal portion of a human eye.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical console or a standalone light source.

Note also that, as described herein, an inferior end, segment, or portion of a component refers to the end, segment, or portion that is beneath or lower such as a bottom or underside of a tissue or structure. Conversely, a superior end, segment, or portion of the component refers to the end, segment, or portion that is above or higher such as a top or topside of the tissue or structure.

Further note that, as described herein, a medial end, segment, or portion of a component refers to the end, segment, or portion that is closest to an inside or a midline of a body. On the other hand, a lateral end, segment, or portion of the component refers to the end, segment, or portion that is closest to an outside of the body or furthest from the midline of the body.

As used herein, the term “about” may refer to a +/−10% variation from the nominal value. It is to be understood that such a variation can be included in any value provided herein.

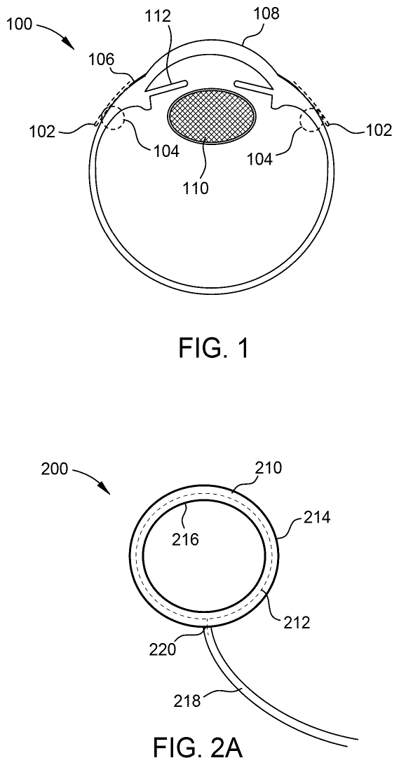

FIG. 1 illustrates a representation of a human eye, according to embodiments described herein. As depicted in FIG. 1, the representation 100 illustrates a fornix 102, a pars plana 104, a sclera 106, a cornea 108, a lens 110, and an iris 112. The fornix 102 is a recessed portion of the conjunctiva that is formed where the eyelids interface with the sclera 106.

The pars plana 104 is a region within the ciliary body commonly utilized to access the posterior segment during vitreoretinal surgical procedures (e.g., to remove portions of the vitreous humor). This access is typically achieved via cannulas which are inserted into small incisions made in the pars plana 104 during the procedure. For instance, the cannulas may control an intraocular pressure and/or mitigate trauma to ocular tissue from inserting/removing various surgical instruments.

The sclera 106 is the white/opaque fibrous tissue that is the structural layer of the outer eye and forms its round shape. The sclera 106 extends from the cornea 108 (the transparent front surface of the eye) to the optic nerve at the back of the eye. The cornea 108 covers the iris 112 which is the colored part of the eye that controls the size of the pupil. The pupil allows light into the eye which the lens 110 focuses on the retina at the back of the eye. The retina includes photoreceptor cells which convert the light into signals for visual perception.

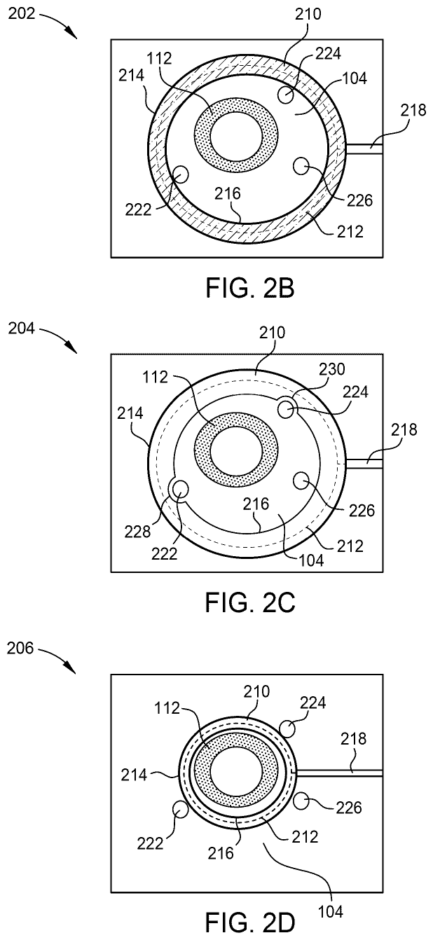

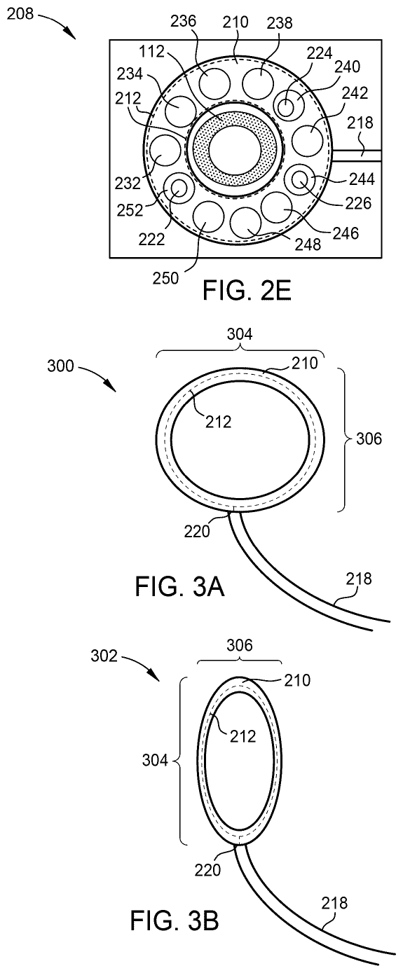

FIGS. 2A, 2B, 2C, 2D, and 2E illustrate top views of transscleral illuminator examples, according to embodiments described herein. FIG. 2A depicts a top view of a transscleral illuminator example 200. As shown, example 200 includes a base 210 and an optical fiber 212 disposed in the base 210. In example 200, the base 210 has a continuous circular geometry. In other examples, the base 210 has a continuous elliptical geometry. In some examples, the base 210 does not have a continuous geometry. In various embodiments, the base 210 has an outer diameter 214 and an inner dimeter 216. For instance, the optical fiber 212 is illustrated to be disposed between the outer diameter 214 and the inner diameter 216. In some embodiments, a surface of the base 210 is configured to maintain a relative orientation between the base 210 and the sclera.

In some examples, the base 210 is manufactured from an optically transparent material. In other examples, the base 210 (or portions of the base 210) may be manufactured from materials configured to diffuse light, reflect light, absorb light, etc. For example, the base 210 (or portions of the base 210) can be “frosted” (e.g., via sandblasting, etching, etc.). In some embodiments, the base 210 is manufactured from a flexible material such that a shape/geometry of the base 210 is generally adjustable. In other embodiments, the base 210 is manufactured from a solid/rigid material such that the shape/geometry of the base 210 is generally not adjustable. Examples of materials for the base 210 include plastics such as thermoplastic polymeric materials, elastomeric materials, metallic materials, combinations thereof, etc.

The optical fiber 212 is manufactured from a material configured to transmit light. For example, the optical fiber 212 may be manufactured from silica (e.g., glass), sapphire, other crystals or ceramics, polymers (e.g., plastic), combinations thereof, etc. In various embodiments, a portion of the optical fiber 212 (e.g., a distal end of the optical fiber 212) may be shaped or modified to increase or decrease an angle of illumination (e.g., a size an illumination field) for light transmitted out from the portion of the optical fiber 212. For example, the portion of the optical fiber 212 can be shaped to maximize the angle of illumination for light transmitted out from the portion of the optical fiber 212 in a “chandelier” configuration. Note that, although a single optical fiber 212 is illustrated and described, the optical fiber 212 may comprise a plurality of optical fibers, such as a bundle of optical fibers, as described in further detail below.

In one or more embodiments, a portion of the optical fiber 212 can be disposed in a jacket 218. In example 200, the jacket 218 forms an interface 220 with the outer diameter 214 of the base 210. In some embodiments, the jacket 218 extends from the interface 220 to a light source connector that connects to a light source. The light source can be integrated into a surgical console or the light source may be a standalone light source. The light source transmits light into the optical fiber 212 at an end of the optical fiber 212 (e.g., a proximal end of the optical fiber 212), and the light is transmitted out from the optical fiber 212 and through the base 210 to illuminate a surgical site.

In various embodiments, the light source is capable of producing white light for transscleral illumination (e.g., a xenon light source). In one or more embodiments, the light source is capable of producing adjustable colors (wavelengths) of light for transscleral illumination (e.g., an RGB (Red Green Blue) light source). In some examples, the light source includes one or more light emitting diodes configured to emit a particular color of light (e.g., red light, green light, blue light, etc.). In these examples, the color of light transmitted out from the optical fiber 212 and through the base 210 and the sclera 106 into the inner eye is adjustable by adjusting a relative intensity of the one or more light emitting diodes. In some embodiments, adjusting the color of the light transmitted through the sclera 106 and the retina may compensate for natural filtering occurring via the transmission through the sclera 106 and/or the retina (e.g., the retinal pigment epithelium).

Notably, adjusting the color of the light in this manner may facilitate diagnostics and imaging in addition or alternative to illumination of a surgical site. It should also be appreciated that, in some embodiments, the transscleral illuminator can be used to illuminate the surgical site in addition to other illumination sources (e.g., endo-illuminators). However, in other embodiments, the transscleral illuminator may be utilized as an alternative to the other illumination sources in order to facilitate bimanual surgical procedures (e.g., simultaneous use of forceps and scissors).

FIG. 2B depicts a top view of a transscleral illuminator example 202. Example 202 includes a representation of the base 210 disposed over the pars plana 104. In example 202, the base 210 is illustrated as having a masked portion which absorbs light transmitted out from the optical fiber 212 and prevents this light from being transmitted through a superior surface of the base 210. For example, the masked portion prevents light from impairing a view of the base 210 from above the base 210.

Example 202 also includes cannulas 222-226 which are inserted in incisions made in the pars plana 104. In some embodiments, the cannulas 222-226 include valves that are configured to maintain an intraocular pressure of the eye during a vitreoretinal surgical procedure. For instance, the cannulas 222-226 facilitate access to the posterior segment of the eye while minimizing trauma to the pars plana 104 as various surgical instruments are inserted/removed and manipulated to perform the procedure.

As shown in FIG. 2B, the cannulas 222-226 are disposed within the inner diameter 116 of the base 210. For instance, cannula 222 can be adjacent to the inner diameter 116, and cannula 224 may also be adjacent to the inner diameter 116. In one or more embodiments, the cannula 222 and/or the cannula 224 may be configured to maintain a position of the base 210 relative to the pars plana 104 during the vitreoretinal surgical procedure. For example, the cannula 222 and/or the cannula 224 may be configured to prevent undesirable movements of the base 210 relative to the pars plana 104 during the procedure. In various embodiments, the base 210 has a geometry that is based on the cannulas 222-226 inserted in the pars plana 104. In an example, the base 210 has a geometry that is based on a location of the cannulas 222-226 when the cannulas 222-226 are inserted in the pars plana 104 in order to perform the procedure.

Notably, the jacket 118 extends laterally in example 202 (e.g., the iris 112 is an iris of a patient's left eye) in order to minimize interference with the procedure. However, in some examples, the jacket 118 extends medially (e.g., the iris 112 is an iris of a patient's right eye). In one or more embodiments, the base is rotatable 210 such that the jacket 118 is extendable laterally or medially regardless of whether the iris 112 is an iris of a patient's left eye or the patient's right eye.

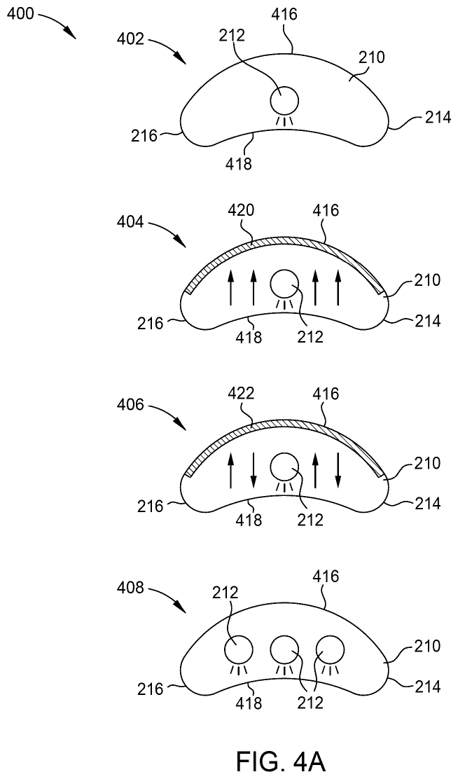

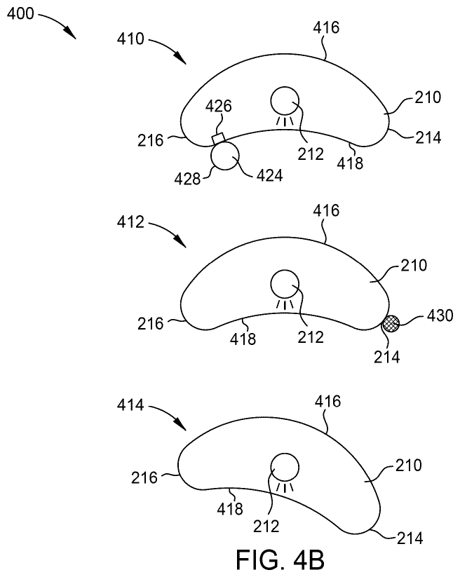

In certain embodiments, the base 210 may be disposed within the cannulas 222-226 or disposed around the cannulas 222-226 such that no portions of the cannulas 222-226 abuts or contacts a portion of the base 210. In some examples, no portions of the cannulas 222-226 abut or contact a portion of the base 210 if the base 210 is disposed in the fornix 102 or adjacent to the fornix 102. In various embodiments, other features/components (e.g., other than the cannulas 222-226) can be used to maintain a position of the base 210 during a surgical procedure. For instance, some of these other features/components are described below with reference to FIG. 4B.

FIG. 2C depicts a top view of a transscleral illuminator example 204. Like example 202, example 204 includes a representation of the base 210 disposed over the pars plana 104. As shown in example 204, the base 210 includes a first recess 228 and a second recess 230. In one or more embodiments, the first recess 228 is configured to interface with the cannula 222 and the second recess 230 is configured to interface with the cannula 224. In some embodiments, the first recess 228 is configured to partially contain the cannula 222 and the second recess 230 is configured to partially contain the cannula 224. In an example, the first recess 228 and the cannula 222 and/or the second recess 230 and the cannula 224 may be configured to maintain the position of the base 210 relative to the pars plana 104 during the vitreoretinal surgical procedure. Note that in certain embodiments, the base 210 may include less or more recess than illustrated in FIG. 2C.

FIG. 2D depicts a top view of a transscleral illuminator example 206. Example 206 includes a representation of the base 210 disposed over the pars plana 104. As shown in FIG. 2D, the base 210 is disposed between the cannulas 222-224. In some embodiments, the cannula 222 may be adjacent to the outer diameter 214 and the cannula 224 can be adjacent to the outer diameter 214. In an example, the cannula 226 may be adjacent to the outer diameter 214. In various embodiments, the cannulas 222-224 may be configured to maintain the position of the base 210 relative to the pars plana 104 during the procedure.

FIG. 2E depicts a top view of a transscleral illuminator example 208. Like example 206, example 208 includes a representation of the base 210 disposed over the pars plana 104. In example 208, the base 210 includes apertures 232-252. The optical fiber 212 is illustrated as being disposed within the apertures 232-252, and the optical fiber 212 is also illustrated as being disposed around the apertures 232-252. In an example in which a single optical fiber 212 is disposed in the base 210, the single optical fiber 212 may be disposed within or around the apertures 232-252. In examples in which multiple optical fibers 212 are disposed in the base 210, at least one optical fiber 212 can be disposed within the apertures 232-252 and/or at least one optical fiber 212 may be disposed around the apertures 232-252.

As shown in FIG. 2E, the apertures 232-252 are identically sized and evenly spaced around the base 210. However, in some examples, the apertures 232-252 are not identically sized and/or the apertures 232-252 are not evenly spaced around the base 210. Although the example 208 includes 11 of the apertures 232-252, it is to be appreciated that in other examples the base 210 includes more or less than 11 of the apertures 232-252.

In one or more embodiments, the apertures 232-252 may be configured as a guide to indicate a position within the pars plana 104 to incise and insert the cannulas 222-224. In some embodiments, the apertures 232-252 are configured to enclose or contain one of the cannulas 222-226. As depicted in FIG. 2E, the cannula 222 is disposed in aperture 252, the cannula 224 is disposed in aperture 240, and the cannula 226 is disposed in aperture 244. In one or more embodiments, the cannula 222 and the aperture 252, the cannula 224 and the aperture 240, and/or the cannula 226 and the aperture 244 can be configured to maintain the position of the base 210 relative to the pars plana 104 during the vitreoretinal surgical procedure. In some examples, the apertures 232-252 may be sized to allow a threshold amount of movement of the base 210 relative to the pars plana 104 during the procedure. In one example, the apertures 232-252 can have aperture diameters which are a length that is about 1.5 to 2.0 times a length of an outer diameter of the cannulas 222-226. In another example, the apertures 232-252 may have aperture diameters which are a length that is less than about 1.5 times or greater than about 2.0 times the length of the outer diameter of the cannulas 222-226.

FIGS. 3A and 3B illustrate top views of optical fiber interface orientation examples, according to embodiments described herein. FIG. 3A illustrates a top view of an optical fiber interface orientation example 300. In example 300, the base 210 has a continuous elliptical geometry such that a first outer diameter 304 of the base 210 is a different length than a second outer diameter 306 of the base 210. As shown in FIG. 3A, in some embodiments, the interface 220 is located at a midpoint of the first outer diameter 304.

FIG. 3B illustrates a top view of an optical fiber interface orientation example 302. Example 302 includes the base 210 having the continuous elliptical geometry with the first outer diameter 304 and the second outer diameter 306. As depicted by FIG. 3B, in various embodiments, the interface 220 is located at a midpoint of the second outer diameter 306.

FIGS. 4A and 4B illustrate cross-sectional views of transscleral illuminator examples, according to embodiments described herein. FIG. 4A illustrates cross-sectional views of transscleral illuminator examples 402-408. Example 402 includes the base 210, the optical fiber 212, the outer diameter 214, and the inner diameter 216. Example 402 also includes a superior surface 416 of the base 210 and an inferior surface 418 of the base 210. In various embodiments, the inferior surface 418 is configured to interface with an external portion of the human eye (e.g., the fornix 102, the pars plana 104, the sclera 106, etc.). In some embodiments, a geometry of the inferior surface 418 includes a curvature which generally corresponds to a curvature of the sclera 106 or other outer surface of a patient's eye. As shown in FIG. 4A, edges of the outer diameter 214 and the inner diameter 216 can be rounded to prevent the edges from “catching” on a surgical glove or the sclera 106 or other surface of the patient during a procedure.

Example 404 includes the base 210, the optical fiber 212, the outer diameter 214, the inner diameter 216, the superior surface 416, and the inferior surface 418. In example 404, the superior surface 416 is illustrated to include a masked portion 420. In one or more embodiments, the masked portion 420 is configured to absorb light transmitted out from the optical fiber 212 to prevent the light from being transmitted through the superior surface 416. For example, the masked portion 420 prevents light transmitted out from the optical fiber 212 from impairing a surgeon's vision during a procedure.

In example 406, the superior surface 416 is illustrated to include a reflective portion 422. In some embodiments, the reflective portion 422 is a mirror or a reflective coating which is configured to reflect light transmitted out from the optical fiber 212. For example, the reflective portion 422 is configured to reflect the light transmitted out from the optical fiber 212 such that the reflected light is transmitted through the inferior surface 418 to illuminate an internal portion of the eye (e.g., the posterior segment). In various embodiments, the reflective portion 422 is configured to maximize an amount of light transmitted through the sclera 106 and into the surgical site.

Example 408 includes the base 210, the outer diameter 214, the inner diameter 216, the superior surface 416, and the inferior surface 418. In example 408, the optical fiber 212 is illustrated as including multiple optical fibers 212. In some embodiments, multiple optical fibers 212 are disposed in the base 210 in order to increase an amount of light transmitted through the inferior surface 418 and the sclera 106 relative to an example in which a single optical fiber 212 is disposed in the base 210. Although three optical fibers 212 are depicted in example 408, it is to be appreciated that, in some examples, two optical fibers 212 are disposed in the base 210, four optical fibers 212 are disposed in the base 210, more than four optical fibers 212 are disposed in the base 210, etc. In certain examples, the multiple optical fibers 212 are arranged in a bundle of optical fibers.

FIG. 4B illustrates cross-sectional views of transscleral illuminator examples 410-414. Example 410 includes the base 210, the optical fiber 212, the outer diameter 214, the inner diameter 216, the superior surface 416, and the inferior surface 418. In example 410, the inferior surface 418 is illustrated to include a stop 424. In one or more embodiments, the stop 424 is configured to maintain a relative position between the base 210 and the external portion of the human eye (e.g., the fornix 102, the pars plana 104, the sclera 106, etc.) during a procedure. For example, the stop 424 increases an amount of friction between the external portion of the human eye and the inferior surface 418 in order to prevent undesirable movements of the base 210 during the procedure. In some examples, the stop 424 increases the amount of friction between the external portion of the human eye and the inferior surface 418 by increasing a surface area of the base in contact with the external portion of the human eye from a first surface area 426 to a second surface area 428. For example, the second surface area 428 is greater than the first surface area 426. Additionally or alternatively to the stop 424, the inferior surface 118 may have a variety of geometries (e.g., features added to the inferior surface 118, portions removed from the inferior surface 118, etc.) configured to maintain the relative position between the base 210 and the external portion of the human eye during the procedure.

Example 412 includes an absorbent material 430. In various embodiments, the absorbent material 430 is disposed around the outer diameter 214 and configured to maintain a relative position between the base 210 and the external portion of the human eye during a procedure. For example, the absorbent material 430 may include a thermoplastic, a superabsorbent polymer, etc. In some embodiments, in order to maintain the relative position between the base 210 and the external portion of the human eye during the procedure, the absorbent material 430 absorbs fluid from the external portion of the human eye which may be naturally occurring and/or a result of irrigation during the procedure. For instance, absorbing the fluid generates an attractive force between the absorbent material 430 and the external portion of the human eye which prevents relative movements between the base 210 and the external portion of the human eye.

Example 414 includes the base 210, the optical fiber 212, the outer diameter 214, the inner diameter 216, the superior surface 416, and the inferior surface 418. In example 414, the inferior surface 418 includes a curvature and/or rotation which generally corresponds to (e.g., matches) a curvature of an external portion of the human eye (e.g., an outer globe of the human eye). In one or more embodiments, the inferior surface 418 includes a curvature and/or rotation which generally corresponds to (e.g., matches) the curvature of the sclera 106.

However, in some embodiments, the inferior surface 418 includes a curvature and/or rotation which generally does not correspond to (or nearly corresponds to) the curvature of the sclera 106. For example, this curvature and/or rotation of the inferior surface 418 can deviate from the curvature of the sclera 106 by a threshold amount such that a difference between the curvature and/or rotation of the inferior surface 418 and the curvature of the sclera 106 is configured to maintain a relative position between the base 210 and the external portion of the human eye during a procedure. In some example embodiments, the difference between the curvature and/or rotation of the inferior surface 418 and the curvature of the sclera 106 generates a force of friction that is configured to maintain the relative position between the base 210 and the external portion of the human eye (e.g., the fornix 102, the pars plana 104, the sclera 106, etc.) during the procedure.

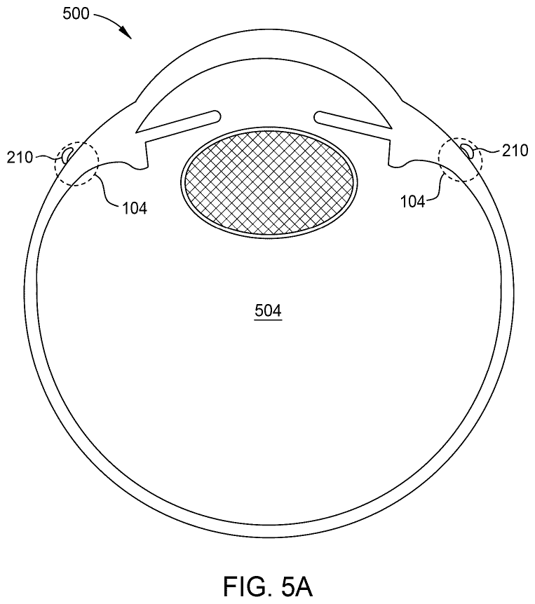

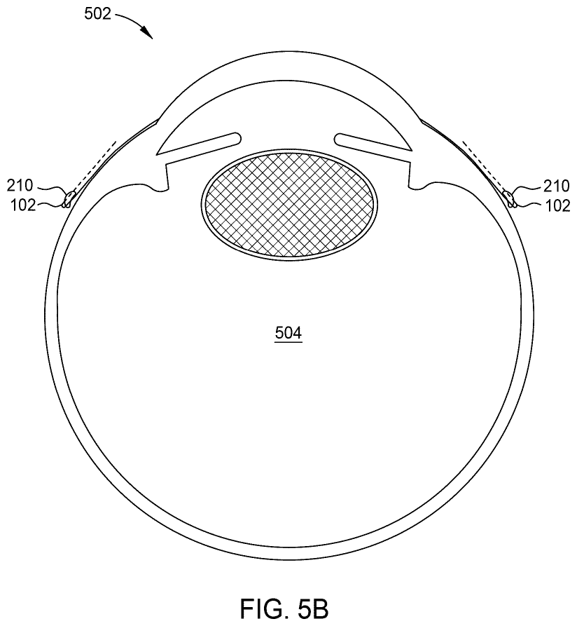

FIGS. 5A and 5B illustrate cross-sectional views of transscleral illuminator examples relative to a representation of a human eye, according to embodiments described herein. FIG. 5A illustrates a cross-sectional view of a transscleral illuminator example 500. In example 500, the base 210 is disposed over the pars plana 104 in order to illuminate an internal portion 504 of the human eye. FIG. 5B illustrates a cross-sectional view of a transscleral illuminator example 502. As shown in FIG. 5B, the base 210 is disposed over the fornix 102 in order to illuminate the internal portion 504 of the human eye.

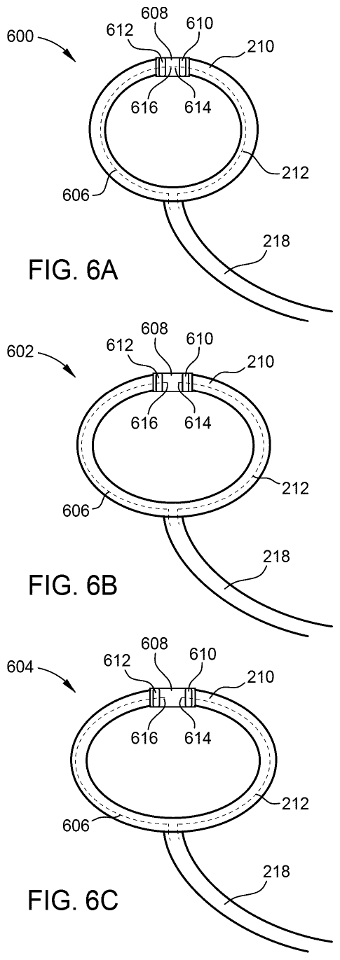

FIGS. 6A, 6B, and 6C illustrate a first example of an adjustable geometry of a transscleral illuminator, according to embodiments described herein. FIG. 6A illustrates a fully compressed base 600. As depicted in FIG. 6A, the fully compressed base 600 includes the base 210 and the optical fiber 212 as well as an additional optical fiber 606. In some embodiments, the additional optical fiber 606 is also disposed in the base 210. The base 210 is illustrated to include an expansion sleeve 608. In various embodiments, the expansion sleeve 608 includes a first friction device 610 (e.g., a first O-ring) and a second friction device 612 (e.g., a second O-ring). For example, the first friction device 610 and the second friction device 612 are configured to temporarily maintain an adjustable size of the base 210 as the fully compressed base 600 by applying a force of friction to portions of the base 210 and portions of the expansion sleeve 608. For instance, a distal end 614 of the optical fiber 212 is illustrated to be disposed in the expansion sleeve 608 and a distal end 616 of the additional optical fiber 606 is also illustrated to be disposed in the expansion sleeve 608. In some embodiments, the additional optical fiber 606 is configured to transmit additional light through the base 210 and the external portion of the human eye to illuminate the internal portion of the human eye.

FIG. 6B illustrates a partially expanded base 602. As shown in FIG. 6B, the partially expanded base 602 includes the base 210, the optical fiber 212, the additional optical fiber 606, and the expansion sleeve 608. In one or more embodiments, a surgeon or a technician can expand the base 210 from the fully compressed base 600 to the partially expanded base 602 by applying a force to a portion of the base 210 in a lateral direction. For example, applying the force to the portion of the base 210 in the lateral direction overcomes the force of friction applied to the portions of the base 210 and the portions of the expansion sleeve 608 by the first friction device 610 and the second friction device 612. After overcoming the force of friction applied to the portions of the base 210 and the portions of the expansion sleeve 608 by the first friction device 610 and the second friction device 612, further application of the force to the portion of the base 210 in the lateral direction by the surgeon or the technician expands the base 210 by decreasing an amount of the base 210 that is disposed within the expansion sleeve 608. In some embodiments, the first friction device 610 and the second friction device 612 are configured to temporarily maintain the adjustable size of the base 210 as the partially expanded base 602 when the surgeon or the technician ceases the further application of the force to the portion of the base 210 in the lateral direction. For instance, a force of friction applied to other portions of the base 210 and other portions of the expansion sleeve 608 by the first friction device 610 and the second friction device 612 is configured to temporarily maintain the adjustable size of the base 210 as the partially expanded base 602.

FIG. 6C illustrates a fully expanded base 604. As depicted in FIG. 6C, the fully expanded base 604 includes the base 210, the optical fiber 212, the additional optical fiber 606, and the expansion sleeve 608. In various embodiments, the surgeon or the technician may expand the base 210 from the partially expanded base 602 to the fully expanded base 604 by applying the force to the portion of the base 210 in the lateral direction and overcoming the force of friction applied to the other portions of the base 210 and the other portions of the expansion sleeve 608 by the first friction device 610 and the second friction device 612. In one or more examples, overcoming the force of friction applied to the other portions of the base 210 and the other portions of the expansion sleeve 608 expands the base 210 by further decreasing the amount of the base 210 that is disposed within the expansion sleeve 608. In some examples, the first friction device 610 and the second friction device 612 are configured to temporarily maintain the adjustable size of the base 210 as the fully expanded base 602. Notably, by expanding the fully compressed base 600 into the partially expanded base 602 or the fully expanded base 604, the base 210 of the transscleral illuminator is capable of illuminating inner portions of human eyes having diameters ranging from myopic to hyperopic and all sizes in between.

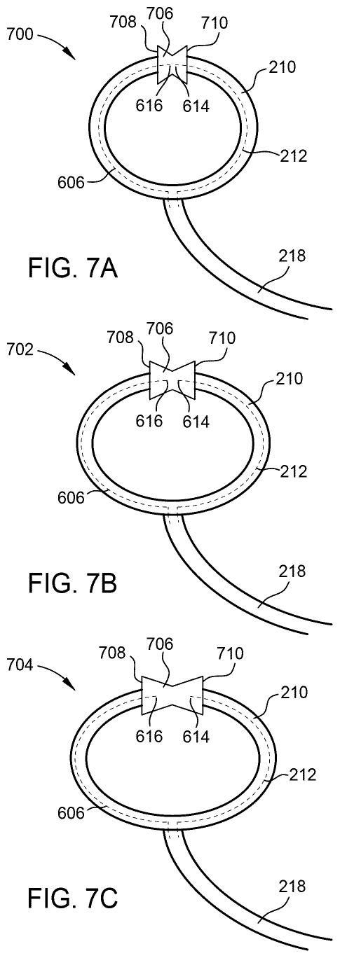

FIGS. 7A, 7B, and 7C illustrate a second example of an adjustable geometry of a transscleral illuminator, according to embodiments described herein. FIG. 7A illustrates a second fully compressed base 700. As shown, the second fully compressed base 700 includes the base 210, the optical fiber 212, the additional optical fiber 606, and an expandable sleeve 706 having a first end 708 and a second end 710. In some examples, the distal end 614 of the optical fiber 212 and the distal end 616 of the additional optical fiber 606 are disposed in the expandable sleeve 706.

FIG. 7B illustrates a second partially expanded base 702. The second partially expanded base 702 includes the base 210, the optical fiber 212, the additional optical fiber 606, and the expandable sleeve 706. In one or more embodiments, it is possible to expand the base 210 from the second fully compressed base 700 to the second partially expanded base 702 by applying a force to the expandable sleeve 706 configured to increase a distance between the first end 708 and the second end 710 (e.g., pulling the first end 708 and the second end 710 apart in opposite directions). In some examples, ends of the base 210 are coupled to portions of the expandable sleeve 706 such that increasing the distance between the first end 708 and the second end 710 is configured to expand the base 210.

FIG. 7C illustrates a second fully expanded base 704. As depicted in FIG. 7C, the second fully expanded base 704 includes the base 210, the optical fiber 212, the additional optical fiber 606, and the expandable sleeve 706. In various embodiments, a surgeon or a technician can expand the base 210 from the second partially expanded base 702 to the second fully expanded base 704 by applying the force to the expandable sleeve 706 that is configured to increase the distance between the first end 708 and the second end 710.

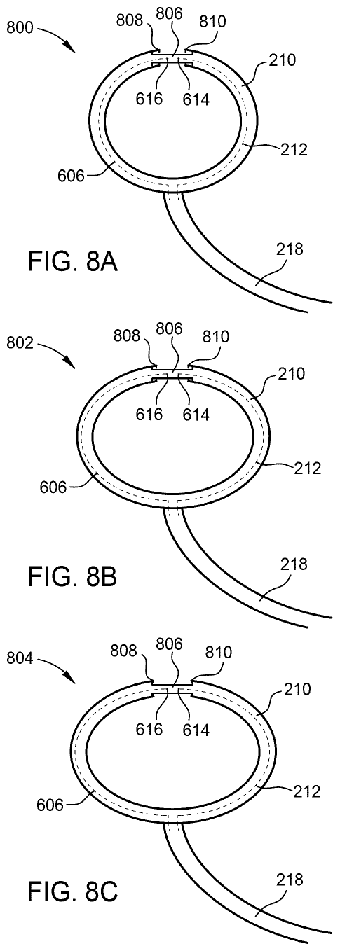

FIGS. 8A, 8B, and 8C illustrate a third example of an adjustable geometry of a transscleral illuminator, according to embodiments described herein. FIG. 8A illustrates a third fully compressed base 800. As illustrated in FIG. 8A, the third fully compressed base 800 includes the base 210, the optical fiber 212, the additional optical fiber 606, an illumination rod 806, a first actuatable end 808, and a second actuatable end 810. For example, the illumination rod 806 is configured to transmit light (e.g., the illumination rod 806 can be manufactured from a same material as the optical fiber 212 and/or the additional optical fiber 606). In some embodiments, the distal end 614 of the optical fiber 212 is disposed in the illumination rod 806 and the distal end 616 of the additional optical fiber 606 is also disposed in the illumination rod 806 such that the optical fiber 212, the illumination rod 806, and the additional optical fiber 606 are configured to transmit light around an entire portion of the base 210.

FIG. 8B illustrates a third partially expanded base 802. The third partially expanded base 802 includes the base 210, the optical fiber 212, the additional optical fiber 606, the illumination rod 806, the first actuatable end 808, and the second actuatable end 810. In various embodiments, a surgeon or a technician can expand the base 210 from the third fully compressed base 800 to the third partially expanded base 802 by applying a force to the first actuatable end 808 and the second actuatable end 810 that is configured to increase a distance between the first actuatable end 808 and the second actuatable end 810. In some examples, the first actuatable end 808, and the second actuatable end 810 are movably coupled to the illumination rod 806 by a snap fit (e.g., a cantilever snap fit) such that increasing the distance between the first actuatable end 808 and the second actuatable end 810 is configured to expand the base 210. For example, at least one embodiment does not prevent the distance between the first actuatable end 808 and the second actuatable end 810 from increasing up to a threshold but prevents the distance between the first actuatable end 808 and the second actuatable end 810 from increasing beyond the threshold.

FIG. 8C illustrates a third fully expanded base 804. As shown, the third fully expanded base 804 includes the base 210, the optical fiber 212, the additional optical fiber 606, the illumination rod 806, the first actuatable end 808, and the second actuatable end 810. In some embodiments, the surgeon or the technician may expand the base 210 from the third partially expanded base 802 to the third fully expanded base 804 by applying the force to the first actuatable end 808 and the second actuatable end 810 that is configured to increase the distance between the first actuatable end 808 and the second actuatable end 810.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A device comprising:
a base having an inferior surface and a superior surface, the inferior surface configured to interface with an external portion of a human eye, the base having a geometry that is based on at least two cannulas configured to be inserted in a pars plana of the human eye; and
an optical fiber disposed in the base, the optical fiber configured to transmit light through the base and the external portion of the human eye to illuminate an internal portion of the human eye.

2. The device of claim 1, wherein the geometry includes an aperture configured to enclose at least one of the at least two cannulas.

3. The device of claim 1, wherein the geometry includes an inner diameter configured to enclose at least one of the at least two cannulas.

4. The device of claim 1, wherein the geometry includes an outer diameter configured to be disposed between the at least two cannulas.

5. The device of claim 1, further comprising an additional optical fiber disposed in the base, the additional optical fiber configured to transmit additional light through the base and the external portion of the human eye to illuminate the internal portion of the human eye.

6. The device of claim 1, further comprising a reflective portion of the superior surface configured to reflect the light to illuminate the internal portion of the human eye.

7. The device of claim 1, further comprising a masked portion of the superior surface configured to prevent transmission of the light through the superior surface.

8. The device of claim 1, wherein the external portion of the human eye includes at least one of the pars plana of the human eye or a fornix of the human eye.

9. The device of claim 1, wherein the geometry is adjustable to increase or decrease an outer diameter of the base.

10. A transscleral illuminator comprising:
a base having a first surface and a second surface, the first surface configured to contact a sclera of a human eye, the base having an adjustable geometry; and
at least one optical fiber partially disposed within the base, the at least one optical fiber configured to transmit light from a light source through the first surface and the sclera to illuminate an inner portion of the human eye.

11. The transscleral illuminator of claim 10, further comprising a recess of the adjustable geometry configured to interface with a cannula.

12. The transscleral illuminator of claim 10, further comprising an aperture of the adjustable geometry configured to enclose a cannula.

13. The transscleral illuminator of claim 10, further comprising a masked portion of the second surface configured to prevent transmission of the light from the light source through the second surface.

14. The transscleral illuminator of claim 10, further comprising a reflective portion of the second surface configured to reflect the light from the light source.

15. The transscleral illuminator of claim 10, wherein the first surface is configured to maintain a relative orientation between the base and the sclera.
